(19) United States
(12) Patent Application Publication
(10) Pub. No.: US 2008/0310583 A1
(43) Pub. Date: Dec. 18, 2008

(54) Cardiopulmonary Screening with Feedback From Analysis to Acquisition

(75) Inventors: Roel Truyen, Turnhout (BE); Michael Grass, Buchholz/Nordheide (DE); Rafael Wiemker, Kisdorf (DE)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/813,367

(22) PCT Filed: Dec. 30, 2005
(86) PCT No.: PCT/IB05/54430

§ 371 Date: Jul. 5, 2007

Related U.S. Application Data
(60) Provisional application No. 60/642,857, filed on Jan. 10, 2005.

(51) Int. Cl. A61B 6/03

(52) U.S. Cl. 378/8; 378/19

(57) ABSTRACT

In a medical screening workflow, a patient is disposed on a patient support (22) of a computed tomography imaging scanner (10). Baseline image data of the patient are acquired using the computed tomography imaging scanner. At least one medical diagnosis operation is performed while the patient remains on the patient support. Baseline image data are processed using a computer-aided lesion detection process performed as a background process during the at least one medical diagnosis operation. Conditional upon the processing detecting at least one lesion: (i) imaging parameters are determined including at least initial and final axial scanner positions for the at least one lesion based on results of the computer-aided lesion detection process; and (ii) an image of the at least one lesion is acquired with the patient remaining on the patient support using the computed tomography imaging scanner configured with the determined imaging parameters.

The following relates to the medical arts. It finds particular application in cardiopulmonary screening for lung cancers, cardiovascular disease, or the like, and will be described with particular reference thereto. More generally, it finds application in the screening, detection, and classification of malignant growths and other medical abnormalities in the heart, lungs, liver, and other vital organs.

Computed tomography imaging provides for rapid and relatively non-invasive cardiopulmonary screening. In the case of cardiac screening, a vascular contrast agent is administered to the patient, followed by acquisition by computed tomography of angiographic data of the heart including vascular contrast enhanced by the vascular contrast agent. The angiographic data acquisition is typically gated with respect to the cardiac cycle to reduce motion blurring, and is acquired with a high x-ray tube current and high spatial resolution. The field of view is usually limited to the heart and associated major vasculature. The resulting reconstructed images are generally analyzed by a cardiologist skilled in interpreting cardiac images.

Lung cancer screening is typically performed at lower resolution, at a lower x-ray tube current, and without the use of a vascular contrast agent. The field of view is large as compared with cardiac screening, typically encompassing most or all of the lungs. In routine screening, the scan parameters are selected to limit radiation exposure of the patient. A radiologist analyzes the acquired low resolution baseline image for suspicious features that may indicate lesions of the lungs. Computer-assisted detection (CAD) may be employed to assist the radiologist in identifying lesions. If these analyses identify one or more suspicious features, the patient is notified and a more intensive imaging work-up is scheduled. The patient returns to the hospital or other imaging center, another baseline scan is performed to re-locate the suspect feature or features, and higher resolution images are acquired of the suspicious feature or features.

Existing cardiopulmonary screening procedures employing computed tomography imaging have certain disadvantages. Existing cardiac screening procedures typically acquire some imaging data from the lungs; however, this data is not used for lung cancer screening. The cardiac screening field of view is insufficient to span the lungs, and moreover the cardiologist is typically not trained to identify lesions of the lungs in computed tomography images.

Existing lung cancer screening procedures do not provide immediate results. If analyses of the low resolution baseline lung scan indicates suspicious features, the patient must schedule a follow-up imaging session. Delays between the baseline imaging and the follow-up session can stress the patient who is uncertain about his or her medical condition; moreover, if a suspicious feature is in fact a malignancy, delaying the follow-up session can allow the cancer time to spread. Still further, at the follow-up imaging session another baseline scan is acquired to re-identify the position of the lesion, which further increases patient radiation exposure.

The following contemplates improved apparatuses and methods that overcome the aforementioned limitations and others.

According to one aspect, a method is provided for detecting and imaging lesions. Baseline image data of a patient on a patient support are acquired using a tomographic imaging scanner. A baseline image is reconstructed while the patient remains on the patient support. Baseline image data are processed using a computer-aided lesion detection process performed as a background process. Conditional upon the processing detecting at least one lesion: (i) imaging parameters are determined including at least initial and final axial scanner positions for the at least one lesion based on results of the computer-aided lesion detection process; and (ii) an image of the at least one lesion is acquired with the patient remaining on the patient support using the computed tomography imaging scanner configured with the determined imaging parameters.

According to another aspect, an apparatus is disclosed for detecting and imaging lesions. The apparatus operates in conjunction with a computed tomography imaging scanner, an image reconstruction processor, an imaging controller configured to cause the scanner to acquire image data, and means for performing at least one medical diagnosis operation. Means is provided for processing acquired baseline image data using a computer-aided lesion detection process performed as a background process during the at least one medical diagnosis operation. Means is provided for determining imaging parameters including at least initial and final axial scanner positions for a lesion based on results of the computer-aided lesion detection process. Means is provided for constructing an imaging sequence based on the determined imaging parameters which when executed by the imaging controller causes the scanner to acquire an image of the lesion.

According to another aspect, a method is provided for detecting and imaging lesions. Baseline image data are acquired spanning at least the heart and part of the lungs of a patient disposed on a patient support using a tomographic imaging scanner. Images of the heart and lung are reconstructed from baseline raw projection image data. The reconstructed heart image of the heart is analyzed for indications of cardiovascular disease. The lung image is processed using a computer-aided lesion detection process to detect lesions of the lungs. The processing is performed as a background process during reconstruction of the image of the heart, analyzing of the reconstructed image, overnight when the reconstruction system of the computed tomography machine is idle, or so forth. An associated user is notified of any detected lesion of the lungs.

One advantage resides in reduced radiation exposure of the patient.

Another advantage resides in reduced time between initial computed tomography imaging and formulation of a medical diagnosis.

Another advantage resides in reduced requirements for scanner time.

Another advantage resides in combining screening for cardiovascular disease with screening for lung cancers.

Another advantage is that the cardiovascular data are usually acquired at a low pitch and therefore have inherently a high redundancy. In combination with the measured electrocardiographic data, a retrospective gated reconstruction of the complete lung can be achieved, which reduces motion artifacts and therefore improves the quality and outcome of the lesion detection.

Yet another advantage resides in enabling a cardiac screening performed by a cardiologist to additionally provide an early warning of possible lesions of the lungs.

Numerous additional advantages and benefits will become apparent to those of ordinary skill in the art upon reading the following detailed description.

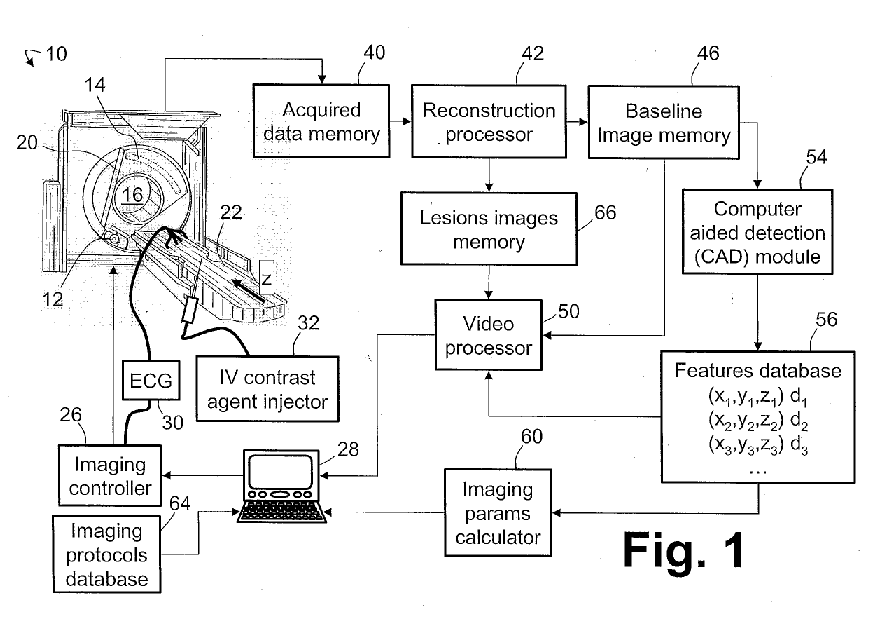

The invention may take form in various components and arrangements of components, and in various process operations and arrangements of process operations. FIG. 1 is only provided for the purpose of illustrating preferred embodiments and is not to be construed as limiting the invention.

FIG. 1 diagrammatically shows a computed tomography system for screening a patient for cardiac disease and/or lung malignancies.

With reference to FIG. 1, a computed tomography imaging scanner 10 includes an x-ray tube 12 and a radiation detector array 14 mounted at opposite sides of an examination region 16 on a rotating gantry 20. A patient support or couch 22 is arranged to support a patient to be imaged and is linearly translatable in a z-direction to move the patient linearly in the z-direction within the examination region 16.

During imaging, the x-ray tube 12 transmits x-rays through the examination region 16. The x-rays are partially absorbed by the patient within the examination region 16, and the transmitted x-rays are measured by the radiation detector array 14 to produce attenuation projection data. The rotating gantry 20 rotates during image acquisition, causing the x-ray tube 12 and detector array 14 to revolve in unison around the patient to acquire imaging data over a range of angular views, such as over a 180° span, a 360° span, or so forth. For screening of vital organs such as the heart, lungs, or liver, the patient is usually arranged on the couch 22 with the patient's axial direction along the z-direction, so that axial slices are acquired.

An imaging controller 26 controls the computed tomography imaging scanner to acquire data over a volume field of view. In some embodiments, the scanner 10 is a multi-slice computed tomography scanner in which the couch 22 is stationary during acquisition of image slices and is stepped in the z-direction between slice acquisitions so as to span the field of view in the z-direction. In other embodiments, the scanner 10 is a cone-beam computed tomography scanner employing a helical scan in which the couch 22 moves linearly during simultaneous rotation of the gantry 16 to produce a helical trajectory of the x-ray tube 12 around the patient.

A user interface 28 allows a radiologist or other user to initiate, monitor, and control the imaging procedure. In the case of cardiac imaging, an electrocardiograph 30 optionally monitors the cardiac cycle, and the imaging is optionally cardiac gated using the electrocardiographic signal or another indicator of the cardiac cycle so as to reduce motion blurring. An intravenous contrast agent injector 32 optionally delivers a contrast agent into the blood prior to imaging. The contrast agent enhances blood contrast in the acquired computed tomographic images. In some procedures, computed tomography imaging is used to monitor flow of contrast agent into and out of a region of interest to provide dynamic flow information useful for isolating vascular blockages, identifying certain characteristic types of lesions, and so forth. In other procedures, the concentration of contrast agent in the blood is maintained in a substantially steady state during the imaging to provide enhanced vascular contrast.

The acquired computed tomography projection data is stored in an acquired data memory 40, and is reconstructed by a reconstruction processor 42 using a filtered backprojection algorithm or other suitable algorithm. For screening, baseline image data of the patient is first acquired, reconstructed by the reconstruction processor 42, and stored in a baseline image memory 46. For lung screening, the baseline image should have a field of view encompassing most or all of the lungs, and typically is acquired using a low power for the x-ray tube 12 and relatively low resolution so as to limit radiation exposure of the patient. Moreover, the baseline lung screening image generally is acquired without a contrast agent. For cardiac screening, the baseline image preferably uses a higher tube power and higher resolution, and employs an intravenous contrast agent. To provide lung screening along with the cardiac screening, the baseline cardiac image preferably also encompasses most or all of the lungs.

The baseline image is used for at least one medical diagnosis operation. In the case of lung screening, the medical diagnosis operation typically includes generating a displayable image of the reconstructed lungs using a video processor 50, and displaying the reconstructed lungs image on the user interface 28 for review by the radiologist or other user. The radiologist can view the baseline lungs image to detect abnormalities. In the case of cardiac screening, the medical diagnosis operation typically includes generating a displayable image of the reconstructed heart using the video processor 50, and displaying the reconstructed cardiac image on the user interface 28 for review and analysis by a cardiologist.

The baseline image is also processed by a computer aided detection module 54 that employs a suitable computer aided detection (CAD) algorithm for detecting lesions of the lungs. The CAD algorithm runs as a background process during the at least one medical diagnosis operation. The CAD algorithm employs suitable image segmentation and feature extraction or classification techniques to identify suspected lesions in the baseline image. Information about any identified suspect features are stored in a features database 56. In the illustrated embodiment, the features database 56 stores position coordinates (x,y,z) of a center of the suspected lesion and a diameter coordinate (d) indicative of the size of the lesion.

In the case of lung screening where the medical diagnosis operation includes displaying the reconstructed lung image, the video processor 50 uses the information in the features database 56 to identify the suspected lesions (if any) on the displayed reconstructed lung image. For example, the identification can be by overlaying an arrow pointing to the lesion onto the displayed reconstructed lung image, or by overlaying a circle drawn around the lesion.

An imaging parameters calculator 60 determines a proposed scan protocol and imaging parameters including at least initial and final axial scanner positions for acquiring a higher resolution image of the lesion. The imaging parameters are determined based on results of the CAD processing stored in the features database 56. For example, in some embodiments the initial position is set at the z-coordinate of the position (x,y,z) minus a scaling factor times the diameter value (d) and the final position is set at the z-coordinate of the position (x,y,z) plus the scaling factor times the diameter value (d). This gives an axial dimension of the field of view which is twice the scaling factor times the diameter value (d). The scaling factor is selected to ensure that the lesion is fully encompassed by the field of view. For example, a scaling factor of two provides an axial field of view that is four times the diameter value (d), which should encompass irregularities of the lesion and include some surrounding tissue. The imaging parameters calculator 60 optionally also determines other imaging parameters such as a helical pitch (corresponding to a linear speed of movement of the couch 22 for a constant rotation rate of the gantry 20) or an x-ray tube current.

The proposed protocol and imaging parameters are selected for a suitable re-scan of the lesion providing improved image characteristics such as higher image resolution. The proposed protocol can be accepted or the re-scan can be custom configured by the user from a suitable lesion imaging protocol from an imaging protocols database 64. The selected lesion imaging protocol typically has some user-selectable parameters, although all parameters may be proposed based on stored information regarding user preferences. Typically, the lesion imaging protocol calls for a high tube current and high resolution, and the field of view is limited to the lesion and surrounding tissue. If the selected imaging protocol calls for administering an intravenous contrast agent, the user is suitably prompted to do so using the intravenous contrast agent injector 32.

During the performing of the at least one medical diagnostic operation and the background CAD processing, the patient remains on the couch 22. Accordingly, after selection and configuration of the imaging protocol and optional administration of contrast agent to the patient, the radiologist or other user is prompted to execute the selected and configured imaging protocol. After receiving user authorization, the imaging controller 26 executes the lesion imaging protocol to produce acquired imaging data that is stored in the memory 40 and reconstructed by the reconstruction processor 42 to produce a high resolution image of the lesion that is stored in a lesion images memory 66. The high resolution lesion image can be processed by the video processor 50 and displayed on the user interface 28 for diagnostic review by a radiologist, pulmonary specialist, or other appropriate medical person.

In the case of a cardiac screening, the work flow is similar. The cardiac image is displayed and analyzed by the cardiologist to identify indications of cardiovascular disease. The CAD module 54 initiates a re-reconstruction of the complete FOV. It operates in the background to analyze the reconstructed lungs so as to detect and determine position, size and optionally other parameters pertaining to suspected lesions. Information about any suspected lesions are is stored in the features database 56. If one or more suspected lesions are identified, then once the cardiologist completes the cardiac analysis the video processor 50 switches over to a display of the reconstructed lungs including identification of the suspected lesions by overlaid arrows, circles, or the like.

Suitable imaging parameters are computed by the imaging parameters calculator 60, an imaging protocol is selected from the imaging protocols database 64. The user can authorize further scanning by authorizing the proposed protocol and parameters or select a different protocol and parameters.

However, in the case of a cardiac scan, the user is sometimes a cardiologist, who may not be qualified to authorize a re-scan of the lungs. Moreover, the cardiac scan is typically acquired at high resolution and high x-ray tube power so as to provide a detailed image for cardiac analysis. Accordingly, in some cardiac screening work flow embodiments, the display of the reconstructed lungs including identified suspected lesions serves as notification to the cardiologist of suspected lesions of the lungs. The cardiologist can then forward this information to appropriate medical personnel such as a radiologist, a pulmonary specialist, or the like for diagnosis. The information about suspected lesions can be forwarded, for example, by printing out the display of the reconstructed lungs with the identified suspected lesions, or by sending this information electronically to the radiologist or pulmonary specialist via a hospital local area network. Because the cardiac image is high resolution, the pulmonary specialist can enlarge the region around suspected lesions using the user interface 28 or another user interface to diagnose the suspected lesion or lesions. A cardiac scan is typically limited to the transverse slab in which the heart is located. If lesions or nodes are seen in this region of the lungs, the cardiologist may authorize a lower power lung screening scan for the rest of the lungs. In this manner, the radiologist is provided with a full set of lung screening images which are high resolution adjacent the heart and low resolution elsewhere.

The illustrated embodiment of FIG. 1 is an expository example that is amenable to many modifications. For example, the imaging controller 26, video processor 50, memories, 46, 66, databases 56, 64, and processors 42, 54, 60 are shown in FIG. 1 as separate components; however, some or all of these components are optionally integrated as software and/or hardware components of a unitary computer that also embodies the user interface 28. Moreover, while cardiac and pulmonary screening are described herein, the work flows disclosed herein are also amenable to screening other vital organs such as the liver.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1. A method for detecting and imaging lesions, the method comprising:
acquiring baseline image data of a patient disposed on a patient support using a tomographic imaging scanner;
reconstructing the baseline image data while the patient remains on the patient support;
processing baseline image data using a computer-aided lesion diagnostic process performed as a background process; and
conditional upon the computer-aided diagnostic processing detecting at least one lesion:
(i) determining imaging parameters including at least initial and final axial scanner positions for the at least one lesion based on results of the computer-aided lesion detection process, and
(ii) acquiring an image of at least one lesion with the patient remaining on the patient support using the tomographic imaging scanner configured with the determined imaging parameters.

2. The method as set forth in claim 1, wherein the acquiring of baseline image data includes acquiring image data spanning at least most of the lungs, and the computer-aided lesion detection process is applied to detect lesions of the lungs.

3. The method as set forth in claim 2, wherein the acquiring of baseline image data further includes acquiring image data spanning the heart, and further including:
reconstructing an image of the heart from a portion of the base line image data spanning baseline image data; and
analyzing the reconstructed image of the heart for indications of cardiovascular disease.

4. The method as set forth in claim 3, wherein the acquiring of baseline image data includes:
administering a vascular contrast agent; and
acquiring high resolution computed tomography angiographic image data spanning the heart and part of the lungs.

5. The method as set forth in claim 3, wherein the acquiring of baseline image data includes:
acquiring cardiac cycle-gated imaging data.

6. The method as set forth in claim 2, wherein the acquiring of baseline image data includes:
acquiring baseline image data using a lower radiation power compared with a higher radiation power used in the acquiring of the image of the at least one lesion.

7. The method as set forth in claim 6, wherein the acquiring of baseline image data is performed without administering vascular contrast agent, and the method further includes:
administering a vascular contrast agent prior to the acquiring of the image of the at least one lesion.

8. The method as set forth in claim 2, wherein the imaging parameters determined for each lesion further include:
determining a helical pitch and an x-ray tube current.

9. The method as set forth in claim 2, wherein the determining of imaging parameters for the at least one lesion includes:
selecting a stored lesion imaging protocol having user-selectable parameters including at least initial and final axial scanner positions; and
automatically setting at least some proposed parameters of the stored lesion imaging protocol based on the results of the computer-aided lesion detection process.

10. The method as set forth in claim 9, wherein the selecting of the stored lesion imaging protocol includes:
displaying a reconstructed image of the lesion to an associated user; and
receiving from the associated authorization to commence a follow-up scan using the stored lesion imaging protocol.

11. The method as set forth in claim 1, wherein the acquiring of baseline image data includes acquiring image data spanning at least most of the liver, and the computer-aided lesion detection process is applied to detect lesions of the liver.

12. The method as set forth in claim 1, wherein the determining of initial and final axial scanner positions for the at least one lesion includes:
determining a position of a center of the lesion; and
determining a width of the lesion in the axial direction.

13. The method as set forth in claim 1, wherein the acquiring of the image of the at least one lesion includes:
acquiring the image conditional upon receiving authorization to acquire the image from an associated user.

14. The method as set forth in claim 1, further including:
performing at least one medical diagnosis operation including reconstructing baseline image data into a reconstructed baseline image of the patient, displaying the reconstructed baseline image to an associated user, and identifying the detected at least one lesion in the display.

15. A tomographic imaging scanner including a processor programmed to perform the method of claim 1.

16. An apparatus for detecting and imaging lesions, the apparatus operating in conjunction with a tomographic imaging scanner, an image reconstruction processor, and an imaging controller configured to cause the scanner to acquire image data, the apparatus comprising:
means for processing acquired baseline image data using a computer-aided lesion detection process performed as a background process during the at least one medical diagnosis operation;
means for determining imaging parameters including at least initial and final axial scanner positions for a lesion based on results of the computer-aided lesion detection process; and
means for constructing an imaging sequence based on the determined imaging parameters which when executed by the imaging controller causes the scanner to acquire an image of the lesion.

17. The apparatus as set forth in claim 16, further including:
means for receiving a user authorization, the constructed imaging sequence being executed by the imaging controller responsive to receipt of the user authorization.

18. A method for detecting and imaging lesions, the method comprising:
acquiring baseline image data spanning at least the heart and a portion of the lungs of a patient using a tomographic imaging scanner;
reconstructing an image of the heart from baseline image data;
reconstructing an image of the portion of the lungs;
analyzing the reconstructed image of the heart for indications of cardiovascular disease;
processing the lung image data using a computer-aided lesion detection process to detect lesions of the lungs, the processing being performed as a background process; and
notifying an associated user of any detected lesion of the lungs.

19. The method as set forth in claim 18, wherein the analyzing of the reconstructed image of the heart for indications of vascular disease includes:
displaying the reconstructed image of the heart to the associated user.

20. The method as set forth in claim 18, wherein the background computer-aided lesion detection process is performed during at least one of the reconstructing of the image of the heart and the analyzing of the reconstructed heart image.

21. A tomographic imaging scanner including a processor programmed to perform the method of claim 18.
